(19) United States
(12) Patent Application Publication
(10) Pub. No.: US 2011/0218432 A1
(43) Pub. Date: Sep. 8, 2011

(54) In Vivo Molecular Imaging

(75) Inventor: Tumay O. Tumer, Beverly Hills, CA (US)

(73) Assignee: NOVA R&D, INC., Riverside, CA (US)

(21) Appl. No.: 13/022,543

(22) Filed: Feb. 7, 2011

Related U.S. Application Data
(60) Provisional application No. 61/301,806, filed on Feb. 5, 2010.

(51) Int. Cl. A61B 6/00; G01T 1/20

(52) U.S. Cl. 600/431; 250/363.01

(57) ABSTRACT

SPECT pixel detectors could be mounted onto moving subjects, such as small rats, to allow live scanning and/or imaging of moving subjects in their natural environment. Solid state monolithic pixel detectors are used for compactness and portability. The pixel detector is designed to be directly read out by integrated circuits and could be flip-chip mounted on the integrated circuit. The detector systems can also wirelessly transmit image data to save space and for ease of portability.

This application claims the benefit of priority to U.S. Provisional Application No. 61/301,806 filed Feb. 5, 2010, which is incorporated herein by reference.

### GOVERNMENT RIGHTS NOTICE

This invention was made with U.S. Government support funding SBIR Phase I research. The Government has certain rights in the invention.

### FIELD OF THE INVENTION

The field of the invention is molecular imaging techniques

### BACKGROUND

Radioactive isotope labeling of small molecules, antibodies, and other probes, in conjunction with the development of dedicated imaging equipment, enables in-vivo molecular imaging. In the past, small animal studies required the dissection of subjects and the performance of autoradiography or direct tissue counting of specimens. This methodology precluded the performance of longitudinal studies that investigated interventions in a single animal. For example, in the past, most methods to image gene expression from transplanted “reporter” genes in rodents, primates, and humans required the use of fixed tissue, obtained either from biopsies or after death. Newer methods using luciferase and green fluorescent protein to tag reporter genes are limited in their in vivo application to transparent or small organisms and cannot be directly translated to human applications.

Usually general anesthesia is used in small animal imaging to prevent motion artifacts. Anesthesia is acceptable for some types of animal studies and is used routinely. However, anesthesia has a significant impact on some body functions such as brain function and is a concern in the interpretation of SPECT and PET studies, especially of neurotransmitter systems. Anesthesia also prevents direct correlations of behavior with simultaneously acquired SPECT and PET data. Therefore, SPECT and PET imaging of conscious and awake small animals are important and recently several research studies have been published. For example, “Rat-PET study without anesthesia: anesthetics modify the dopamine D1 receptor binding in rat brain” by S. Momosaki et al., (Synapse 2004, 54:207-13), “Ketamine alters the availability of striatal dopamine transporter as measured by [C-11]beta-CFT and [C-11]beta-CIT-FE in the monkey brain” by H. Tsukada et al. (Synapse 2001, 42:273-80), “Miniaturized Head-Mounted PET for Conscious Rodent Brain Imaging” by P. Vaska et al. (IEEE Trans. Nucl. Sci. 2004, 51:2718-22), and “RatCAP: a small, head-mounted PET tomograph for imaging the brain of an awake rat” by C. Woody et al. (Nucl Instr Meth A 2004, 527:166-70), each of which is incorporated herein by reference. RatCAP, for example, is a PET imaging system to eliminate motion artifacts as well as the undesirable effects of anesthesia via a miniaturization of the scanner and direct attachment to the rat's head. However, the RatCAP system holds the rats head fixed and limits movement of the rat.

These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

US2005/0082486 to Schlyer and US2005/0113667 to Schlyer both teach a PET scanner that is mounted to a rat's head, which allows the rat being scanned to move around instead of necessitating the system to hold the rat's head fixed. Schlyer's PET scanner, however, requires a physical tether to be coupled to the rat, limiting the rat's movement, which prevents the scanner from analyzing the rat's tissue in a more “natural” environment.

### SUMMARY OF THE INVENTION

The inventive subject matter provides apparatus, systems and methods in which one could scan a subject by coupling a detector to the subject, where the detector receives a signal from the subject and an integrated circuit coupled to the detector produces an image from the signal. The detector used is preferably a monolithic pixel detector. As used herein, a “monolithic pixel detector” is a detector with a two-dimensional array of electrodes mounted upon a single monolithic detector substrate. As used herein, a “subject” is any object that could be imaged using a detector. Preferably, the subject is a live animal, such as a mouse or a human, but the subject could be a non-living animal, such as a dead piece of flesh, a live plant, a machine, an instrument or a device without departing from the scope of the invention.

Generally, radiation is introduced into the subject such that particles produced by the radiation are captured by the detector. For example, since the pixel detector is mounted on one side of the subject, a radiation-producing device could be mounted on the opposing side of the subject, and could emit radiation that flows through the subject into the detector. In an alternative embodiment, a radiation-producing substance, such as a radiopharmaceutical, could be introduced to a portion of the subject (typically through injection), which emits radiation in towards the detector. Generally, radiopharmaceuticals produce particles such as x-rays, gamma rays, photons, electrons and/or positrons. Positrons annihilate with an electron to produce at least two photons that can be captured one or more signals when the photon is received by the detector.

The inventive subject matter could comprise multiple monolithic detectors in different configurations around the subject. For example a large detector could be created by positioning multiple monolithic detectors adjacent to one another in a detector array, or a plurality of detectors could be placed adjacent to one another on a harness or in a belt-like fashion to wrap around the subject. In an alternative embodiment, detectors could be coupled to opposing sides of the subject to capture slices of images running through the entire width of the subject.

Each monolithic detector preferably comprises a plurality of channels that receive signals (such as photons) from the radioactive substance introduced to the subject. The detectors preferably also have a collimator coupled to the detector to help focus and/or redirect signals to specific channels.

Signals received by the detector are preferably processed by an integrated circuit coupled to the detector to produce image data from the signal. As used herein, “image data” is any data captured by a detector that could be utilized to produce an image of a portion of the subject, which includes both raw data and a processed image. Preferably, the integrated circuit is directly flip-chip bonded to the detector. As used herein, “flip-chip” bonding means bonding a chip (an integrated circuit) through pixel pads onto matching pixel pads of a detector or a carrier. For example, a chip could be bonded to a detector through pixel pads, or to a carrier through pixel pads or to another chip through pixel pads. If a chip is bonded onto a carrier, a detector or another chip could be bonded onto the other side of the carrier with or without matching pixel pitch and/or geometry. Other bump bonding techniques such as solder, Z-Bond and indium-bump bonding techniques could also be used without departing from the scope of the invention. The weight of both the detector and the integrated circuit is preferably fully supported by the harness such that the subject supports the weight of the detector device. This way, no wires or other machinery coupled to the detector act as a leash or other type of moment restrictor that limits the movement of the subject being scanned.

The image data produced by the integrated circuit is preferably stored on storage memory coupled to the integrated circuit or on storage memory physically separate from the integrated circuit. As used herein, memory that is physically separate from a circuit is memory that is not connected using wires, such that the data must be transmitted using a wireless signal, such as wifi, Bluetooth, or radiofrequency. Any suitable wireless transceiver that transmits image data to a distal wireless receiver could be used for such a purpose. The detection system could also be powered wirelessly as well, for example through an inductive power transmitter physically separate from the detection system.

Where the storage memory is physically coupled to the integrated circuit, the detector may have a port that couples to the storage memory to transmit the image data to a device coupled to the port, but preferably the storage memory is detachable from the system so as to be easily imported into a remote computer.

In another embodiment, the detection system is preferably small and portable and is mounted to the subject using a harness, such as a belt or a piece of clothing. In a preferred embodiment, the harness comprises a helmet or a hat that elastically fits around a head of an animal subject to capture particles such as photons while the animal subject is alive, conscious and moves.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic of infrared transmission maps for exemplary LPB CZT wafers.

FIG. 2 is a perspective view of sample LPB grown CZT material based single detectors.

FIG. 3 is a graph of a sample ²⁴¹Am spectrum.

FIG. 4 are graphs of x-ray responses for the detectors in FIG. 2.

FIG. 5 is a table showing mobility lifetime products for electrons and holes for the detectors in FIG. 2.

FIG. 6 is a graph of a sample ²⁴¹Am spectrum for an exemplary detector.

FIG. 7 is a graph of a ²⁴¹Am spectrum for another exemplary detector.

FIG. 8 is a schematic of an exemplary signal channel.

FIG. 9A-9C are plan views of an exemplary ASIC.

FIG. 10 is a schematic of an exemplary test setup to check chip tolerance.

FIG. 11 is a graph of a sample pulser response

FIG. 12 is a plan view of an exemplary mask layout.

FIG. 13 is a graph of a simulation for the channel of FIG. 8.

FIG. 14 is a graph of input amplifier and gain amplifier responses to a signal input.

FIG. 15 is a graph of a oscilloscope test signal juxtaposed with a trigger signal.

FIG. 16 is a graph showing pulser spectra for various input amplitudes.

FIG. 17 is a graph showing mean values of Gauss functions fitted to the spectra shown in FIG. 16 as a function of input amplitude.

FIG. 18 is a graph showing a ⁵⁷Co-spectrum for a pixel on an exemplary CZT detector array.

FIG. 19 is a plan view of an exemplary collimator.

FIG. 20 is a graph of the linear attenuation coefficient for CZT as a function of gamma ray energy for exemplary CZT detector arrays.

FIG. 21 is a graph showing gamma ray energy absorption as a function of CZT depth thickness for exemplary CZT detector arrays.

FIG. 22 is a perspective view of an exemplary system as used on a NUDE mouse.

FIG. 23A-23D are perspective views of alternative exemplary systems.

FIG. 24A-24B is an exploded view of an exemplary detector system including the monolithic CZT detectors showing the two-dimensional pixels/electrodes (FIG. 24A).

FIG. 25 is a perspective view of an exemplary hybrid monolithic pixel detector showing the bump bonding through indium bumps.

FIG. 26 is a list of exemplary specifications for a CZT pixel detector and ASIC

FIG. 27 is a graph of simulated responses for a preamplifier, differentiator, and lowpass filter.

FIG. 28 is a graph of the noise spectrum at an output of an input amplifier and lowpass filter.

FIG. 29 is a schematic of a circuit board for an exemplary detector system.

FIG. 30 is a schematic of an exemplary circuit board layout.

FIG. 31 is a table of comparisons of dimensions between an exemplary SPECT device and prior art SPECT devices.

### DETAILED DESCRIPTION

One embodiment of the invention comprises a wireless Mini SPECT system for a conscious small animal where the subject being scanned, such as an animal, is free to move. This would allow imaging in a more natural environment. The proposed system could be used for different kinds of small animals such as mice and rats. In addition, since the RatCAP and Mini SPECT are complementary systems both systems could be used together.

Single Photon Emission Computed Tomography (SPECT) and Positron Emission Tomography (PET) utilize molecules labeled with radioisotopes emitting particles such as gamma rays, x-rays, electrons or positrons (positrons then annihilate with electrons to produce pairs of gamma rays) to observe and measure rates of biochemical processes in tissues of living subjects. The results obtained with either technique could be translated to human applications; for example with the same probes (PET & SPECT) or with different isotope forms of the same probes (SPECT). Typically, substrates for intracellular enzymes or ligands for cellular receptors are used as molecular probes. Retention of the molecular probe in target tissues, as a result either of conversion of a labeled enzyme substrate to a “trapped” metabolic product or binding of a labeled ligand to its receptor, could be detected and quantitatively imaged in living subjects with a tomograph. Larger molecules like antibodies could also be labeled with a radioactive probe, enabling researchers to monitor the distribution of the therapeutic treatment as it evolves over time. From the PET side, microPET technology could be used for noninvasive imaging of animals such as mice and larger rodents or animals with a volumetric resolution of about 8 mm³. On the SPECT side, large clinical systems or conventional scintillator based systems could be used for studying small animals, such as with a stationary imager based on a hybrid CdZnTe pixel detector. Detector thicknesses in such a system could be ˜2 mm and the pixel detector and ASIC in such a system may need to be cooled to temperatures between −5° C. and −15° C. to work. Preferably, the detector used has a plurality of pixels multiplexed to a single output and read out in a raster scan.

A key element in a gamma camera or a SPECT system is the detector, which preferably converts the gamma rays emitted from the radiotracers into electronic signals. Important detector properties are dynamic range, quantum efficiency, sensitivity, noise characteristics, linearity, uniformity and the ability to provide high spatial resolution over the required area. First generation detectors employ scintillators to convert gamma rays into visible light, which is subsequently converted into an electronic signal. Because of the intermediate conversion stage, this process is inefficient, which typically leads to higher signal noise. In a pixel detector, the light produced in the scintillator propagates isotropically and, if the detector is thick (to increase the photon interaction probability), the detector could diffuse into adjacent pixels and cause blurring. Optical isolation between the pixels is made to alleviate this problem by building the detector array from single isolated detectors or by cutting slots between the pixels but it increases manufacturing cost and produces signal loss for crystals with very narrow aspect ratio. Direct conversion detectors, so called because they directly convert gamma rays into charge signals (electron-hole pairs), could be used to overcome some of these issues. For example, optical or any other type of isolation will no longer be needed and the detectors can be built from monolithic substrates to form monolithic pixel detectors without any slots or cuts. This is because the electron-hole pairs created by the incident particle such as photons in the detector material is charged and the applied bias field conforms the charge movement into columns and directs them onto the pixels/electrodes.

Another embodiment of the invention comprises compact, portable solid state pixel detectors with a dedicated readout IC (integrated circuit) for high spatial and energy resolution and room temperature molecular imaging which could be used in a larger variety of situations—for example in vivo imaging of small animals. These detectors could be used to develop a compact and a portable detector system which could be mounted on a conscious animal to carry out wireless imaging while the animal is mobile. In other words, the detectors could have one or more wireless transmitters that transmit an image or image data wirelessly. The same detectors could also be used to fabricate a small desktop high resolution imaging system. The detector system could be developed to work as a gamma camera and SPECT. These proposed detectors are expected to fill a growing need for high resolution molecular imaging of conscious and aware small animals with new capabilities.

Direct conversion CdZnTe, CdTe, Si, GaAs, PbI₂, and Se pixel detectors could be used, preferably CdZnTe and CdTe pixel detectors. That these detectors have excellent potential for high quantum efficiency at low and intermediate gamma ray energies (<250 keV). These solid state direct conversion detectors also offer significantly improved spatial and energy resolution compared to scintillation detectors and allow for a more compact design, as no photodiodes or photomultiplier tubes are needed to convert the visible light into electronic signals. CdZnTe (CZT) detectors are also preferred because they have excellent properties and availability. CZT and other solid state detectors could be mounted on custom multi channel mixed signal ASICs, with custom electronics, firmware, and software already embedded in the ASIC. In a preferred embodiment, the proposed detector system could be incorporated into a stationary animal imager.

In another embodiment, a small gamma ray imaging pixel detector could enable a high efficiency, high-resolution instrument for imaging molecular tracers labeled with low and intermediate energy gamma-ray emitting isotopes such as ¹²⁵I, ²⁰¹T1 and ^{99m} Tc. Such an instrument could be produced using a hybrid technology involving pixelated solid-state detector(s) bonded to Application Specific Integrated Circuit(s) (ASICs). The detectors preferably consist of a high-Z, high-density photoconductive materials to achieve high gamma ray quantum efficiency. These detectors directly and precisely convert the absorbed energy to an electric signal. Cd₁₋ₓZnₓTe (CZT, ×˜0.1 typically) is a preferred gamma ray detector material. The ASIC directly and efficiently collects the detector signals, digitizes them, and stores them in a computer as an image matrix for further analysis. Thanks to this hybridization, the resulting instruments could be designed to be very compact in size, making them ideal for wireless in-vivo molecular imaging of conscious small animals. Their compactness could allow individual detector modules to be mounted onto conscious and mobile animals using a harness holding the detectors in place.

Preferably, the detector is coupled to a mixed signal readout ASIC for reading and processing the signals from the 2-D detector array pixels. To minimize detector capacitance and signal noise, the detector array is preferably flip-chip bonded directly onto the ASIC using a gold stud bonding technique. As used herein, “flip-chip” bonding means bonding a chip through pixel pads onto matching pixel pads of a detector or a carrier. For example, a chip could be bonded to a detector through pixel pads, or to a carrier through pixel pads or to another chip through pixel pads. Other bump bonding techniques such as solder, Z-Bond and indium-bump bonding techniques could also be used without departing from the scope of the invention. Additional channels could be supplied on the ASIC to read out signals from the detector cathode and other electrodes, if needed to optimize energy resolution. All detector signals are preferably digitized on the chip or on the circuit board by an analog-to-digital converter (ADC) in order to optimize transmission of the scanned signal.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

The detector and readout chip are preferably mounted on compact electronic circuit boards that could provide regulated power supplies—including high voltage or bias voltage for the detector—digital control and readout of the ASIC, and wireless communication with the host computer. For example, a chip could be bump bonded to a CZT pixel array to make a pixel detector. In a preferred embodiment, the CZT pixel detector has a 32×32 array and has an appropriate steering grid and/or a guard ring. Parallel hole collimators (also diverging and/or converging hole collimators) with an appropriate hole diameter could be used to collect a signal and focus them towards specific pixels on the detector. Preferably, as much of the peripheral circuitry as possible is on the chip to reduce the used real estate on the chip. The pitch of the chip is preferably ≈0.5×0.5 mm² but any other pitch size other than square with any other geometry could be used without departing from the scope of the current invention.

The combined detector electronics system is preferably sized and dimensioned to be mountable on a small animal—for example, less than 25 cm² or even 5 cm², although detectors of other sizes could be used without departing from the scope of the invention. Also larger detectors could be made by placing such detectors side by side to form one-, two- or three-dimensional arrays of such detectors to image the entirety of a small animal or to image larger animals. The detectors could be placed with space in between one another, but are preferably abutted to each other or a portion of one another.

CZT Detectors

High Uniformity CZT Detectors

Cadmium zinc telluride (CZT) could be produced with the High-Pressure Bridgman (HPB) growth method to harvest only poly-crystalline CZT material, or the Low-Pressure Bridgman (LPB) method, or the Travelling Heater Method. Preferably, the detector is made from a large-sized, single-crystalline CZT detector material, for example, a material with a volume of up to or larger than 300 cm³, which could be used to create relatively large volume (>2 cm³) detectors from the material, which have achieved better than 1.7% energy resolution at 662 keV.

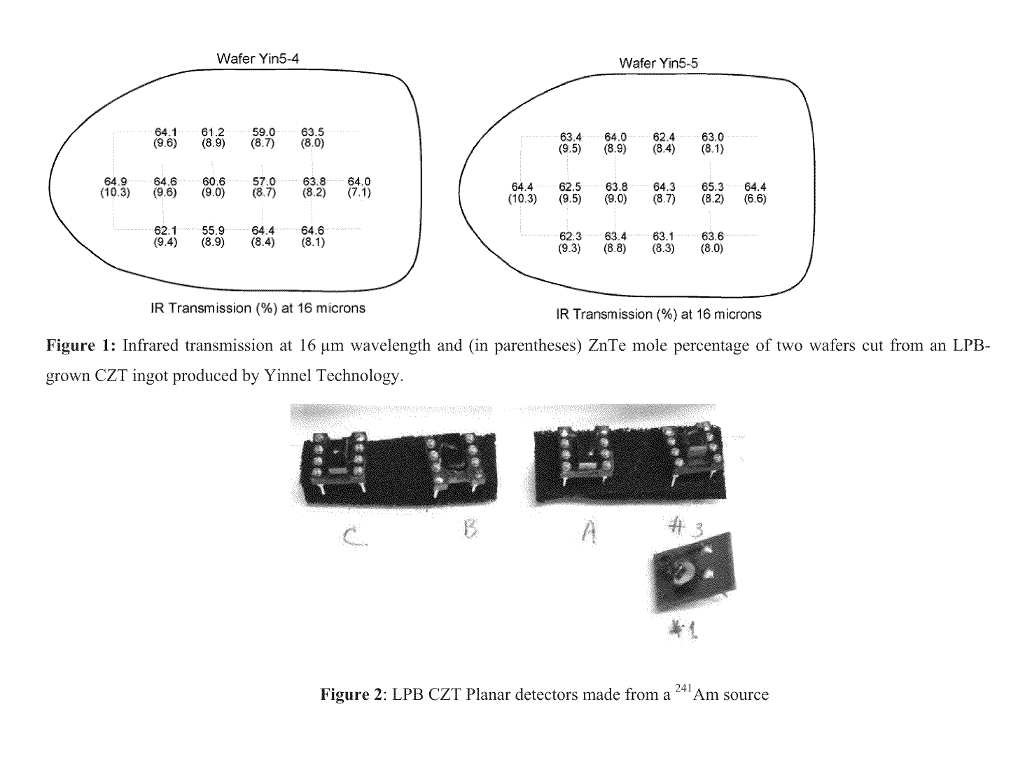

FIG. 1 shows infrared (IR) transmission maps of two LPB CZT wafers, Yin5-4 and Yin5-5. The size of the wafers is 7.5 cm long and 4.5 cm wide. The IR sampling distance between points is 1 cm. In wafer Yin5-4 there are only 3 points where the transmission is less than 60% and in wafer Yin5-5 all the points are above 60%. In a preferred embodiment, the ZnTe mole percentage in the material is distributed uniformly.

Performance of Detectors

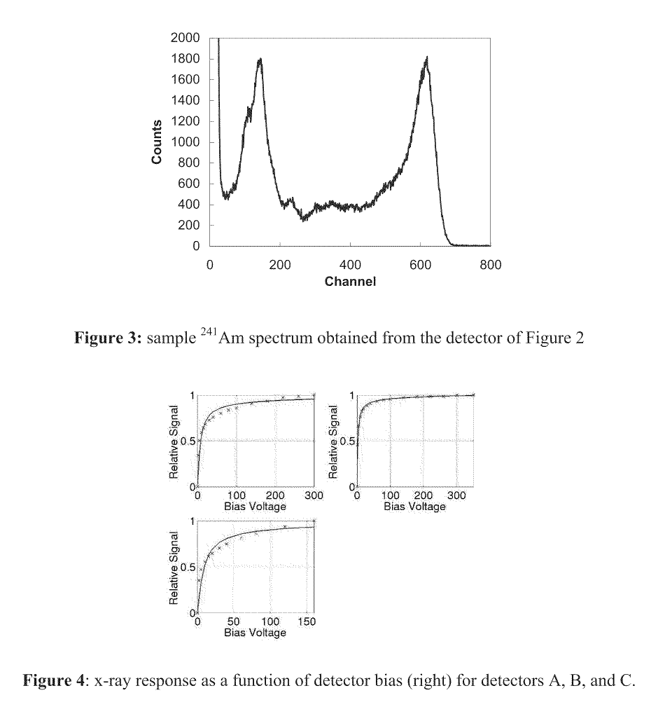

The detector is generally small, made from a single-element (planar) material. FIG. 2 shows sample LPB grown CZT material based single detectors. FIG. 3 shows a ²⁴¹ Am spectrum at room temperature from one of the detectors shown in FIG. 2. About 10% energy resolution was achieved for the 59.5 keV energy peak.

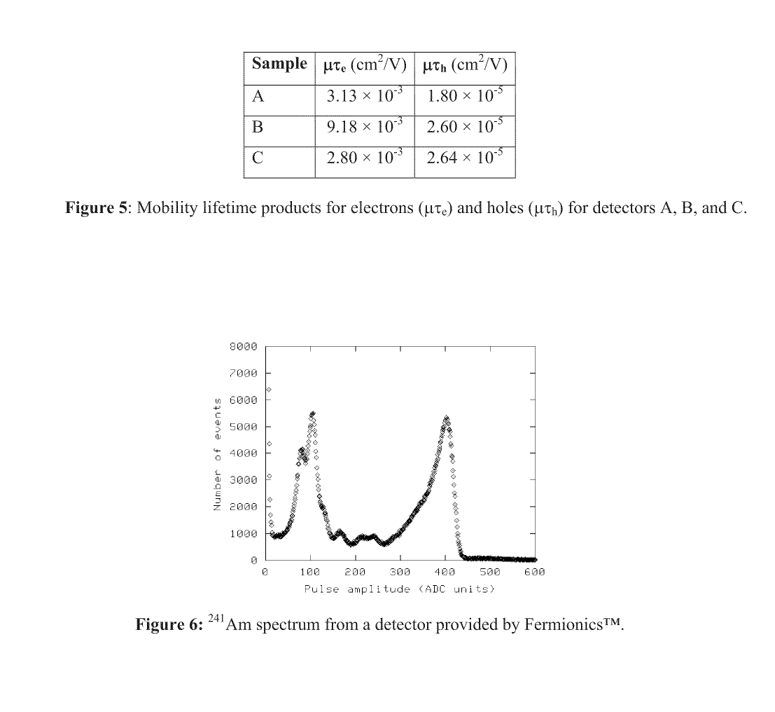

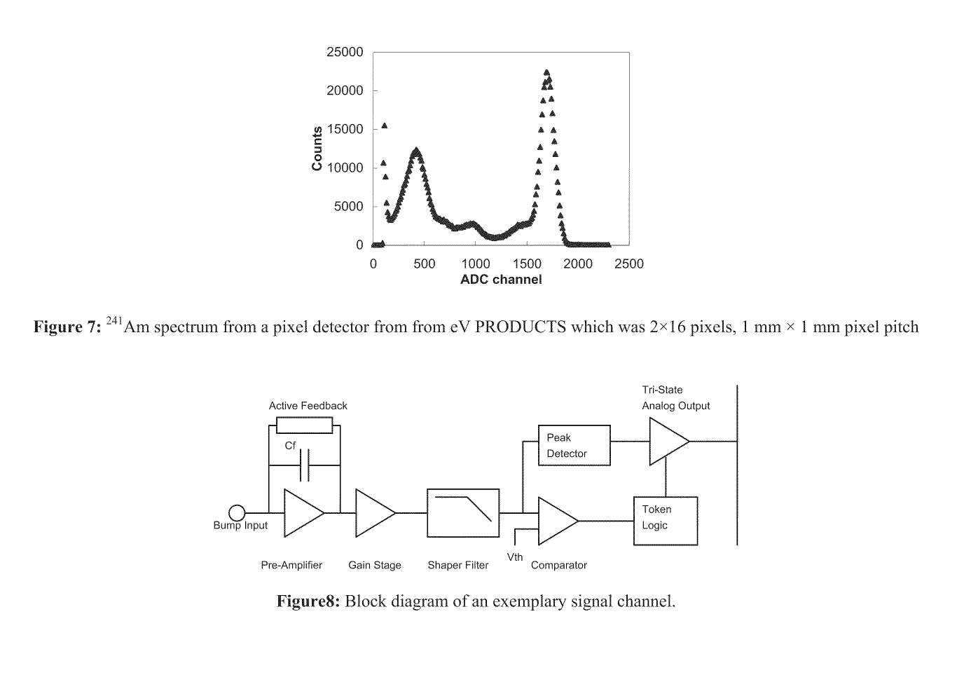

FIG. 4 shows the response of detectors labeled A, B, and C in FIG. 2 to low-energy x-rays as a function of detector bias. The data could then be fit to the Hecht equation to determine the mobility-lifetime (μτ) products for the charge carriers in the CZT, which are tabulated in FIG. 5. Reasonable μτ values could also range from (3-7)×10⁻³ cm²/V for HP-EDG material. FIGS. 6 and 7 show μτ values for two other exemplary detectors. Due to the small-pixel effect, which suppresses the contribution from hole transport, with its major trapping effects, to the detector signal, the 59.5 keV peak shows significantly less low-energy tailing than in all cases of the planar detectors. Further significant improvement in the quality of the spectra could be expected from the use of coplanar grid detectors.

Design and Simulation of a Readout IC for In-Vivo Molecular Imaging

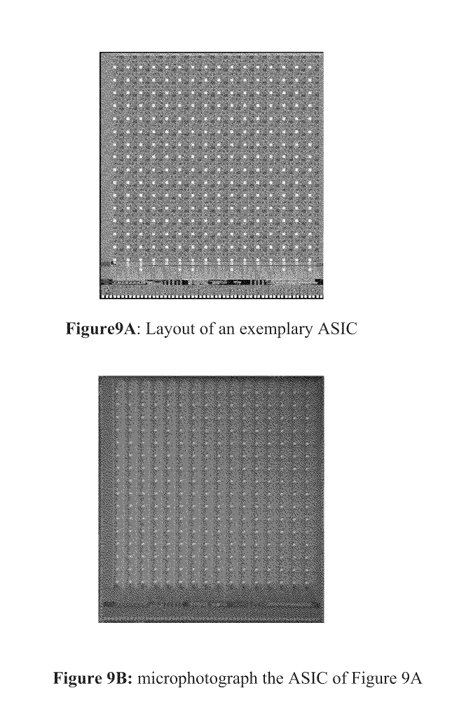

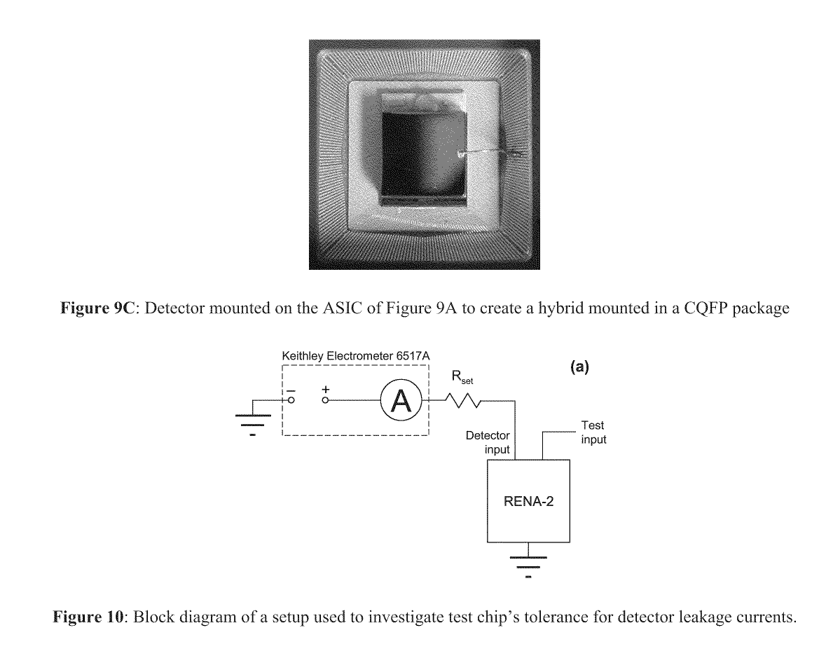

In one exemplary embodiment, an array of 16×16 readout channels (pixels) is integrated on an ASIC, each one featuring a self-resetting charge-sensitive input amplifier, shaper, peak detector, and trigger and readout circuitry. The ASIC is preferably designed for flip-chip bonding to a pixel detector with a pixel pitch of 0.5×0.5 mm². The chip could have two user-selectable input energy ranges, selectable shaping times between 1 μs and 4 μs, and the design target for the input-referred noise is generally 200 e rms or less. FIG. 8 shows a block diagram of a channel signal on the ASIC and FIGS. 9A-9C shows layout diagrams of the detector and the ASIC. FIG. 9C shows a CZT detector array that is bonded onto the chip that hides the chip from view, except for the bottom section with the wire-bond pads, to create a hybrid detector-chip. The detector hybrid is mounted in a CQFP package for testing.

Design Concerns

One of the concerns about flip-chip bonding a solid-state radiation detector directly on top of the readout IC is the handling of the detector leakage currents. Unless decoupling capacitors can be integrated into the chip circuitry, which would raise a number of design issues in itself, the bonding method may not allow for AC coupling between the detector pixels and the input amplifiers. Therefore, the amplifiers need to be able to handle the expected range of leakage currents while maintaining the required input energy range. A carrier with AC coupling could be designed and mounted in between the ASIC and the detector to allow AC coupling between the detector pixels and the input amplifiers on each channel of the ASIC.

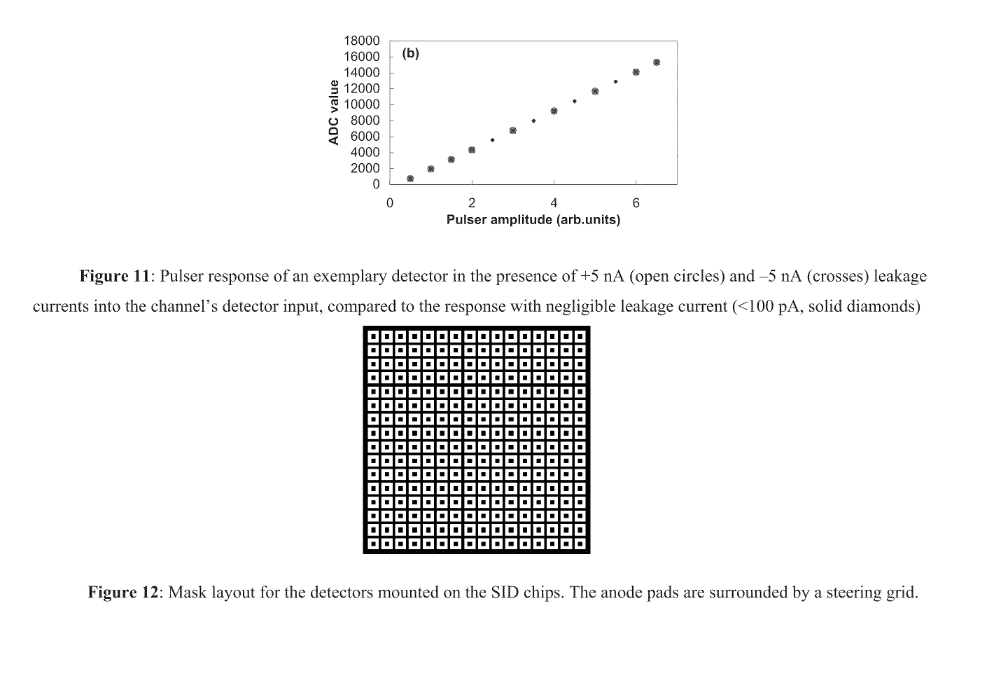

Preferably, a 36-channel detector readout IC could be used to allow DC-coupling to the detectors being read. This readout IC preferably tolerates leakage currents up to ±5 nA. FIG. 10 shows results of an electrometer (Keithley 6517A) test with a built-in voltage source to supply a known current to such a detector's signal inputs through a 500 MΩ resistor. At the same time, a test signal was applied to the selected channel through the chip's test pulse input. The peak detector output was measured as a function of the test signal amplitude for different values of the electrometer current ranging from −5 nA to +5 nA. This response is shown in FIG. 11 for currents of +5 nA, −5 nA, and negligible current (<100 pA). The connection from the electrometer to the chip introduced noise pickup that slightly broadened the distributions of ADC values measured at any given amplitude, compared to the reference data measured without the electrometer connected (not shown). The leakage currents themselves, however, did not have any noticeable effect on the chip's response.

As a more direct study to determine if the detector is suitable is by using three 16×16-pixel CZT detector arrays. An exemplary test was performed using such ships with a pitch of 0.3×0.3 mm². The chip had a purely integrating front end preamplifier and does not tolerate leakage current, and the detector and the chip require significant cooling to low temperatures, −20° C. to −40° C., to work. The mask layout for the detectors is shown in FIG. 12. The detectors were mounted onto three of the chips, using indium-bump bonding. The chip-detector hybrids were mounted on ceramic carriers. An active resistive feedback circuit could also be used to allow the chip to be tolerant to detector leakage current, and therefore the detector can be DC-coupled, which is essential for hybrid pixel detectors. The chip also could have features such as dual energy range, selectable polarity (which would be important to implement cathode readout capability), etc, which is not available in a standard chips. The array size could also be sized to 32×32 or 32×64 or more with proper optimization.

Chip Design and Test Simulations to Show Enablement

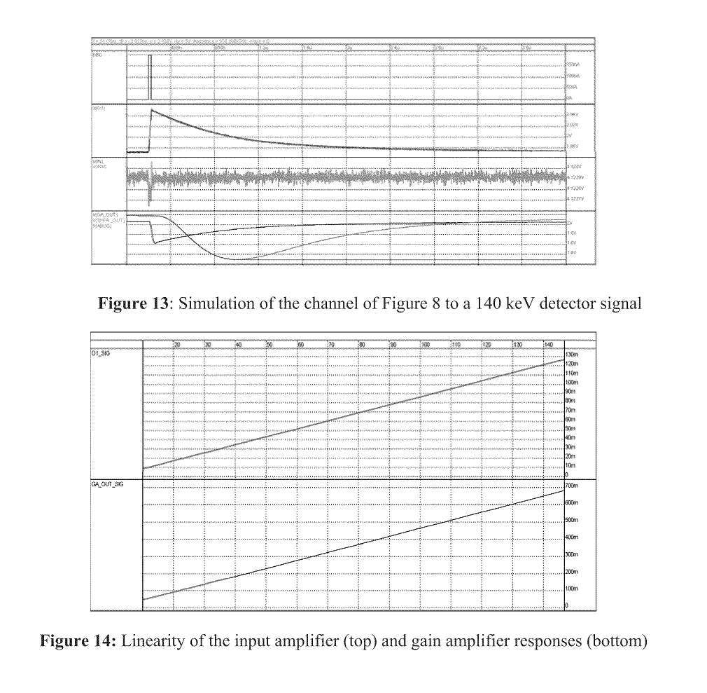

FIG. 13 shows a result of a typical SPICE simulation following a single-signal through different stages of a channel in the channel shown in FIG. 8. The top panel shows a single 25 ns wide current pulse out of the detector; the pulse height corresponds to the interaction of a 140 keV gamma ray in a CZT detector. Noise is added to the input signal in order to study the noise properties of the amplifier chain. The response of the input amplifier is shown in the second panel. The third plot shows the voltage at the input, clearly showing the noise and the input current pulse. The bottom panel shows the outputs of the gain amplifier, the shaper, and the peak detector.

FIG. 14 shows the output amplitudes of the input and gain amplifiers (top and bottom plots, respectively) as a function of the input amplitude. Both curves show excellent linearity over the entire input range covered in the simulation.

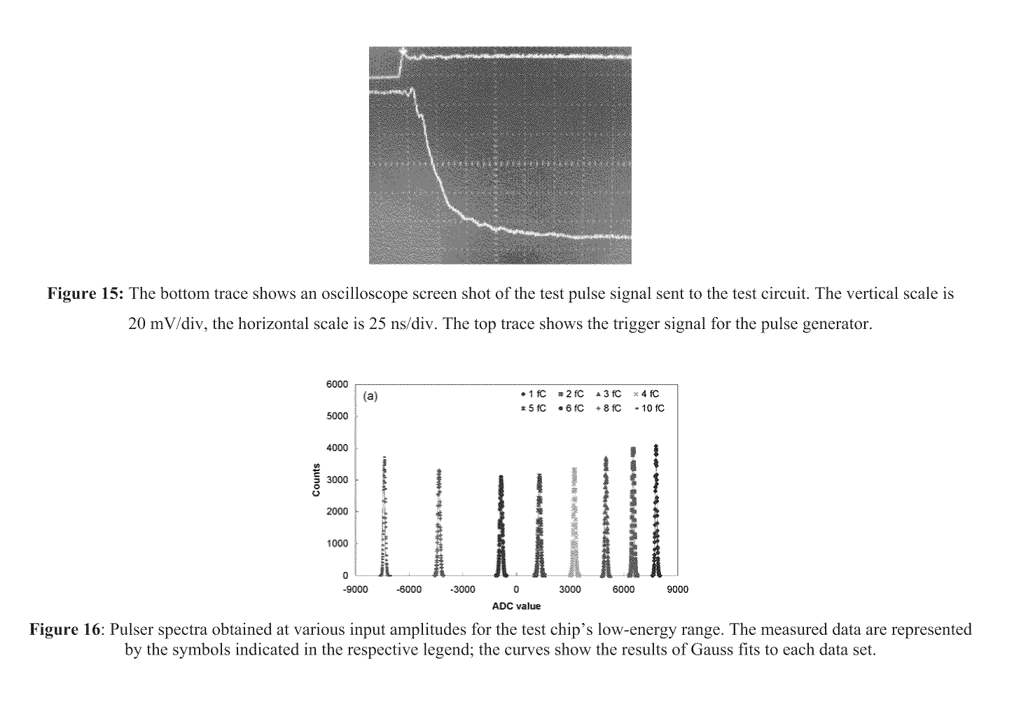

FIG. 15 shows the trigger signal for a pulse generator (top trace) and a sample test pulse (bottom trace) where the integrated circuit under test is installed on a daughter card that plugs into the main test board and provides chip-specific connections and circuits. The test pulses were generated on the test board. The signal rise time (10% to 90%) is approximately 50 ns, the amplitude was measured as 103 mV. Since the signal is applied across a 75 fF capacitor in each DANA IC (integrated circuit) channel, this corresponds to just above 7.5 fC. (The new ASIC described in this application is referred to herein as the new DANA IC or chip.) The pulse amplitude is adjustable between 0 and 20 fC.

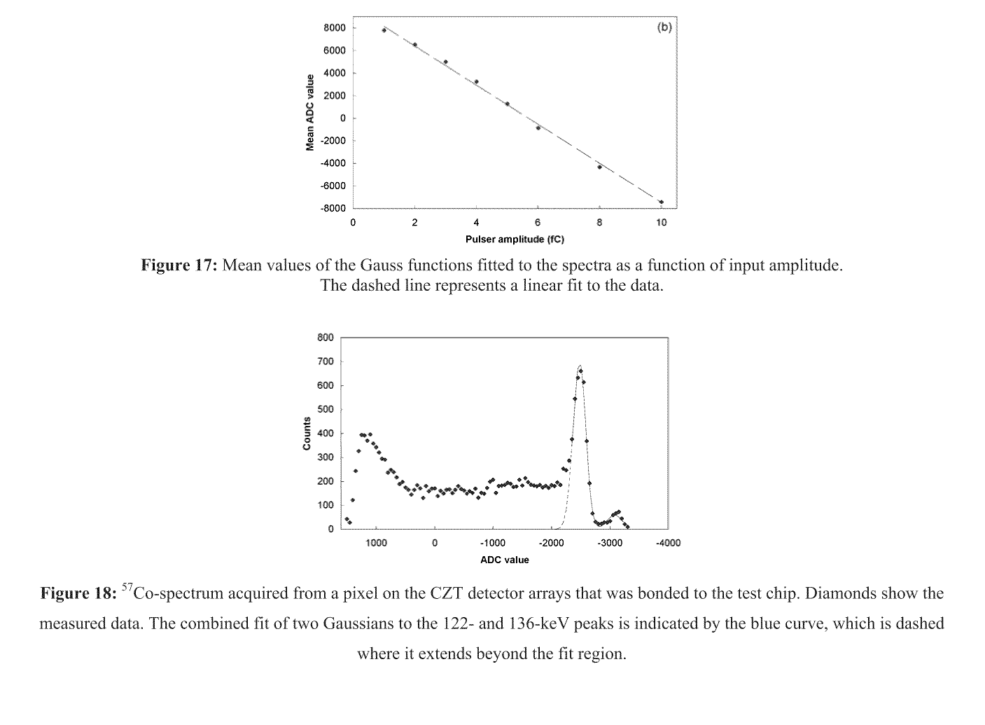

FIG. 16 shows sample spectra obtained from one test channel in the low-energy range, which would be suitable for SPECT imaging (shaping time 2.7 μs, minimum gain setting, comparator threshold 0.2 V relative to the signal baseline). The nominal input amplitude for each spectrum is indicated in the legend. The peak detector output was digitized on the test board by a 14-bit ADC with signed output. For the negative input pulses from FIG. 15, the shaper output polarity is negative as well, so larger amplitudes correspond to lower ADC values. Each spectrum was fitted with a Gaussian distribution; the chi-squared per degree of freedom varied from 0.9 to 3, with most values in the range between 1.2 and 1.6. FIG. 17 shows the mean values of these fit curves as a function of the input pulse amplitude. The results are reasonably close to the straight-line fit, which is indicated by the dashed line. The widths (sigma) of the Gauss curves vary between 200 and 225 electrons (input-referred), except for the lowest and highest input amplitudes, where the width reaches 290 electrons. In the chip's high-energy setting, the range of rms widths is 230 to 275 electrons for all amplitudes except the lowest, where it is 330 electrons.

Hybrid chips were tested by bonding 3 mm thick CZT detector arrays with a matching pixel geometry (16×16 pixels, 0.5 mm×0.5 mm pitch) to three of the test integrated circuits using a gold-stud bumping process, although indium bump bonding could have been used without departing from the scope of the invention. The connection between the detector pixel pads and the corresponding input pads on the chip was accomplished by straight-through vias in the carrier and bonded using an epoxy. FIG. 18 shows a ⁵⁷Co-spectrum acquired from one of the pixels on a test hybrid chip. The energy resolution of the 122- and 136-keV peaks was remarkably good; the double Gauss fit shown gives us a sigma of 2.58 keV for the 122-keV peak and 3.05 keV at 136 keV. If the pixel pitch is small, charge sharing may happen between the pixels and adjacent pixels may produce a signal from the charge received from the actual event pixel. This effect could be mitigated by reading the adjacent pixels and adding all the signals to determine the total charge produced by the incident radiation particle such as a gamma ray or an x-ray photon or a charged particle. The chip could be also designed to disable “hot” channels or pixels. These channels are the ones which produce event triggers from noise and may not contain real events some or most of the time.

Collimator and Circuit Board Design Simulations

There are four basic collimator types for SPECT—namely pinhole, parallel hole, diverging and converging. There are also variations on each collimators such as fine or coarse resolution, low, medium or high energy and divergence/convergence angle, etc. All of these collimators with variations are viable alternatives and have specialized applications. The collimators could also be designed to be fixed in place, removable, movable in and out of a functional position or interchangeable with other collimators. As used herein, a collimator that is “fixed” is one that is coupled using an adhesive while one that is “removable” is one that is coupled using a mechanical coupling that could be removed without damaging either the collimator or the surface the collimator is coupled with.

A pin hole collimator may need careful handling for the proposed portable conscious small animal imager because of the larger distance from the hole to the detector array. It has excellent application and use for small organ or small graft imaging such as mouse kidney, thyroid, small tissue grafts, etc. A parallel collimator (which is the standard collimator for the proposed system as shown in all the illustrations) is the most common in SPECT and has excellent algorithms for calibration and image correction. However, the hole size and wall thickness must be optimized for different applications. Preferably, the hole size is small to make the image high resolution and the collimator thickness small so that the weight of the collimator is low. In an exemplary embodiment, the hole diameter is between 100 microns to 250 microns to match a 500×500 micron detector pitch. However, 500 and 1,000 microns diameter holes are not out of the question and may be important for fast studies, which does not require high spatial resolution.

Converging and diverging collimators may be used for other applications. They are more difficult to use and require carefully developed algorithms for imaging, calibration and image correction. For example, a converging collimator may be used to focus on small organs in the body or parts of the organs with high efficiency, high resolution, which may not be possible with a pin hole camera, while a diverging collimator may be used to image large body areas of the small animal with somewhat lower spatial resolution.

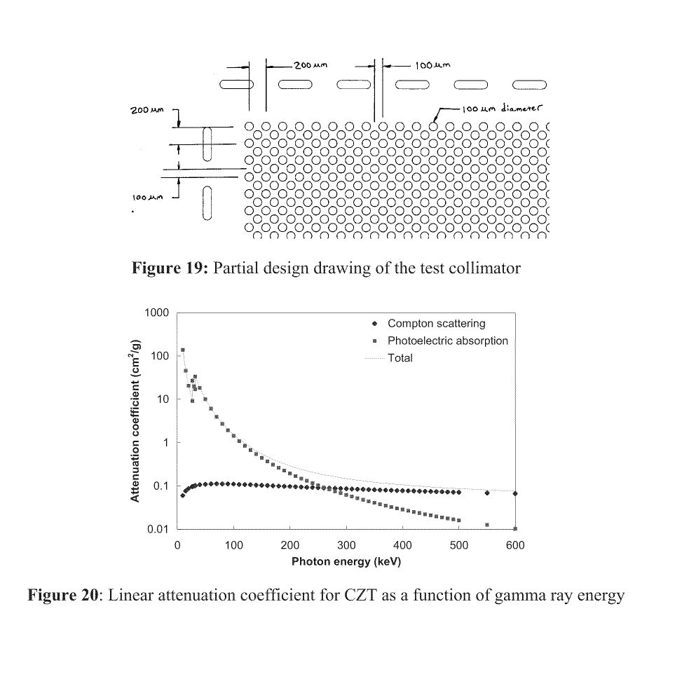

Parallel hole collimators are standard and easier to use and produce images. A partial layout of the test parallel hole collimator is shown in FIG. 19. The collimator had 100 μm diameter bores; the pitch between bores in the same row was 200 μm, with a 100 μm offset between rows, for an overall pitch of ˜140 μm between nearest neighbor bores. The collimator thickness was 1.57 mm. The collimator had good stopping power for 122 keV gamma rays. The collimator was made from 75 μm thick tungsten sheets into which a photolithographic pattern of 260 μm square holes with a pitch of 380 μm had been etched. About 100 sheets were laminated together with an alignment accuracy better than 2 μm to form a 7 mm thick collimator with an aspect ratio of 27:1 and a calculated efficiency of 5×10⁻⁵. The FWHM position resolution achieved with this collimator for a 300 μm diameter ^{99m} Tc source was 450 μm, only about 20% larger than the pixel pitch.

Research Design and Methods

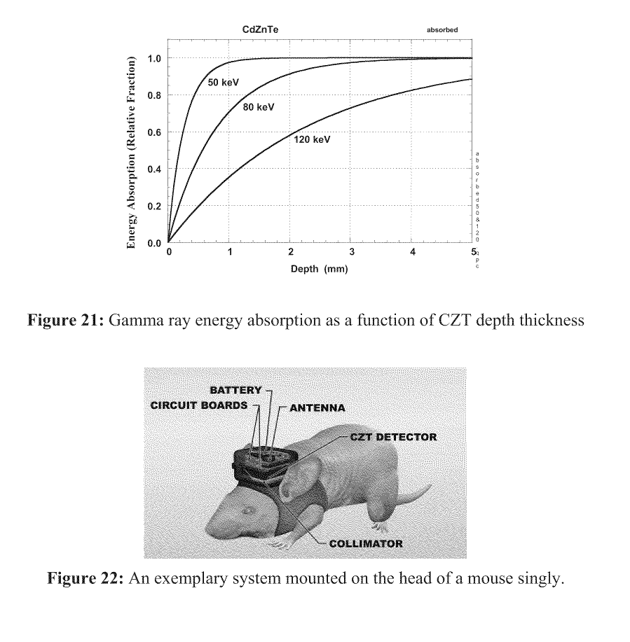

Radiation detector systems for medical imaging, specifically for small animal SPECT imaging, are quite unique and challenging. Most importantly, due to the small size of the animals, excellent spatial resolution, in the sub-mm range, is required in order to resolve the features of interest. We propose a 0.5×0.5 mm² pixel pitch CZT pixel detectors with high resolution collimators. Apart from the obvious requirements for small pixel dimensions and good collimation, achieving good spatial resolution also implies a need for excellent energy resolution. In this case, the energy resolution serves to identify and suppress blurring caused by photon scattering on the path from the animal to the detector, which would otherwise degrade the spatial resolution that can be achieved with a finely pixelated detector. The SPECT energy range is approximately 30 to 200 keV with most useful range with present radioisotopes is from about 50 to 160 keV. Over this energy range, the detector also needs to provide a reasonably high photon detection efficiency in order to acquire images with good contrast in a short time and without excessive radiation load on the animal. FIG. 20 shows the linear photon attenuation coefficient for CZT as a function of gamma energy. From these data, we can calculate the detection probability (quantum efficiency) for gamma rays as a function of energy and detector thickness. Another important information from this figure is the below 250 keV photoelectric absorption dominates. Therefore in the SPECT energy range Compton scattering would be minimal leading to good energy resolution. FIG. 21 shows the energy absorption as a function of depth of CZT detectors. A 2 mm thick CZT detector could preferably have about 85% quantum efficiency for 100 keV gamma rays. For 140 keV photons 3 to 5 mm thick detector could have about 60% to 80% quantum efficiency, respectively. Therefore, a CZT pixel detector with 3 to 5 mm thickness would be sufficient to provide good quantum efficiency and DQE for small animal imaging.

In using the proposed CZT pixel detector for energies up to ˜150-200 keV as a gamma camera or for SPECT imaging, the x-ray flux levels incident on the detector could be limited to values on the order of a thousand photons per second. This limit comes from the collimator efficiency, which for typical collimators would be a few times 10⁻⁴ or less, depending on the desired spatial resolution. These rates are not high for the proposed new DANA chip. However, we plan to design the ASIC to handle data rates of >10,000 events per second per chip to address the potential for using large hole collimators and/or larger animals if applicable.

An additional goal of the proposed project is to design the detector system small enough that it can be mounted on the animal being investigated. Anesthesia or physical restraints during the imaging process can induce physiologic changes in the animals that, in turn, can affect the outcome of the measurements themselves. On the other hand, movement of the animal relative to the detector(s) would cause unacceptable blurring of the image. By mounting the detector on the animal itself, such tedious and difficult corrections would become unnecessary. To achieve this, the detector must be lightweight and compact, since a bulky system would make an animal immobile and also risk causing stress in the animal. In the case of mice, specifically, whose weight of 25-30 g is comparable that of a large (20×20 mm²) detector and collimator, this means that this approach would likely require smaller detectors, approximately 10×10 mm². We plan to compromise and develop the detector with 16×16 mm² area. This size would be applicable to both mice and rats and it is direct extension (4×) of DANA chip, which is 8×8 mm². The weights of a 16×16 mm CZT detector and tungsten collimator with holes are 4.43, 7.4, 4.9 and 10 gr for thicknesses 3, 5, 2 and 4 mm, respectively. The electronics could increase this somewhat. Animal-mounted systems will have to be powered by batteries, so they also need to be designed for low power consumption. A third requirement specific to animal-mounted systems is wireless transmission of the detector data using a commercially available system such as Bluetooth.

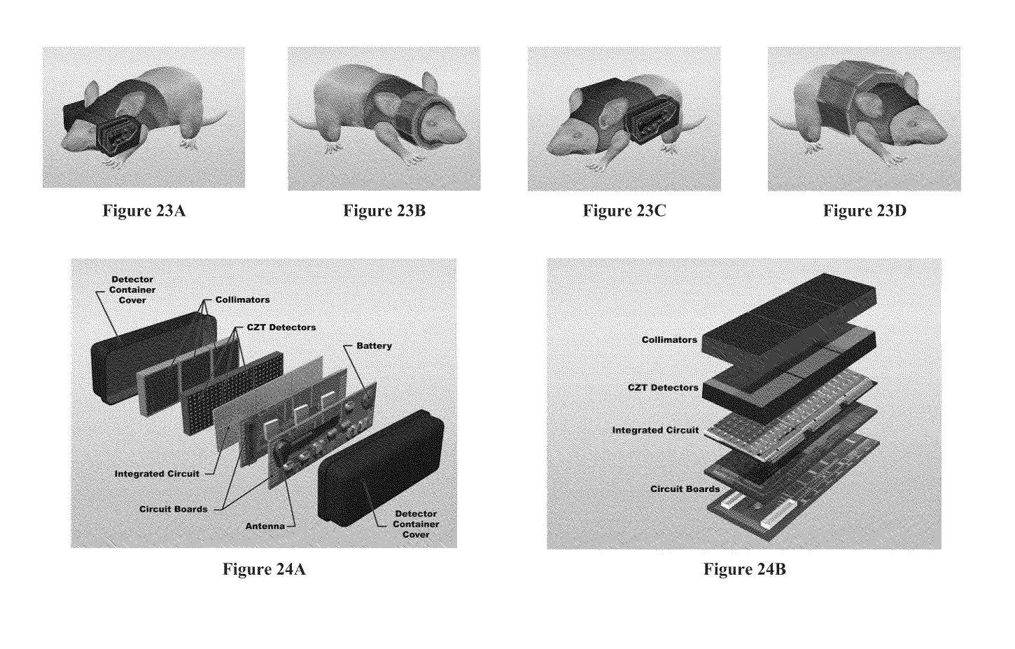

FIG. 22 shows a preferred prototype of the Mini Gamma Camera mounted onto a harness and attached onto a NUDE or a SCID (Severe Combined Immune Deficiency) mouse. FIGS. 23A-23D show several other possible configurations. FIGS. 24A and 24B show exploded views of the Mini Gamma Camera and Mini SPECT with three pixel detectors used side by side. A single or two or more CZT pixel detectors can be used without departing from the scope of the current invention. These solid state detectors have certain advantages such as small compact size and low weight compared to scintillator detectors built from individual detectors formed into two dimensional arrays or slots are sawed to make individual pixels from a single block of scintillator. The solid state detectors are also readout directly because the interaction of the incident particle such as an x-ray or a gamma ray is converted directly into electron hole pairs and detected. In the scintillator based detectors an intermediary detector to convert the light produced by the scintillator into electron hole pairs will be required making them bulky and heavy.

Detector and Readout Requirements for Medical Molecular Imaging Applications

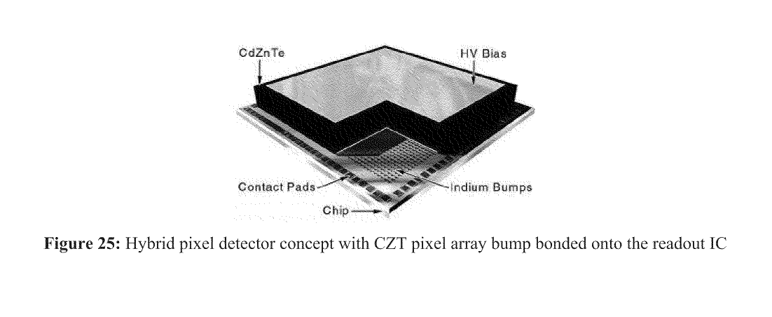

Below are listed some of the most important requirements for the development of a new generation of small size and compact CZT detector system for SPECT applications for molecular imaging using small animals:
1. Hybrid pixel detector design, DANA chip and DANA pixel detector in which the detector is flip-chip bonded onto the readout chip are shown in FIG. 25. This approach minimizes the detector capacitance and allows for a compact design. At the same time, it allows the detector array and the readout electronics to be optimized separately as well as relative to each other to produce the best configuration and capabilities.
2. Tolerance to leakage current, since the hybrid detector design necessitates DC coupling between the detector and readout ASIC. This is achieved in DANA chip by designing a self resetting preamplifier.
3. Room temperature operation, with low noise and high energy resolution.
4. Small pixel pitch, 0.5×0.5 mm², for high spatial resolution.
5. Wide energy range, 30 to 200 keV, for SPECT imaging.
6. Count rate capability of ≧20,000 counts per second per detector.
7. Extra readout channels for the common detector cathode and the steering grid if necessary.
8. Low cost, readily available, uniform and high resistivity CZT from present vendors such as eV Producs, Redlen, Orbotech, Acrorad and Eurorad. Yinnel wafers can be processed into CZT pixel arrays by Aguila.
9. CZT detectors with thickness from 3-5 mm. Larger thickness detectors may also be used without departing from the scope of the invention.

Detector and Readout ASIC Specifications

As discussed above a new ASIC and improved CZT pixel detectors could be used to develop an instrument for medical molecular imaging. Defining the specifications for these key components is a an important step in such a work program.

A summary of the CZT detector and ASIC specifications is given in FIG. 25. The new DANA chip has the following major differences compared to the DANA chip.
1. An array of 32×32 pixels with about 0.5×0.5 mm² pitch instead of DANA chip's 16×16 array with same pitch.
2. Lower noise at each pixel.
3. High dynamic range, 250 keV for CZT, with low energy threshold, about 20 keV. This should be designed to cover most radiopharmaceuticals, from about 30 keV to 200 keV.
4. A second energy range 20 keV to 3 MeV to detect higher energy photons and particles.
5. Lower and adjustable power to compromise between high energy resolution and low power operation. The low power operation is important for running on small batteries and dissipating the heat quickly and easily.
6. Incorporating as much of the peripheral electronics on chip as possible, such ADC, FIFO, logic, buffer, power converter, registers, etc. These circuitry that will go onto the new chip will be decided when the initial design of the peripheral circuitry is ready. This is to achieve smallest possible electronics real estate to produce a compact portable detector that will mount onto of the small animal for imaging while the animal is conscious and moving. The wireless system will be external and based on a commercial standard such as Bluetooth
7. Readout capability for the steering grid and/or the guard ring.
8. Integrating and/or self resetting charge sensitive preamplifier.
9. Making the current into the preamplifier and other sections of the circuitry adjustable.
10. Selectable signal polarity and/or selectable channel input polarity.
11. Sparse readout capability to allow to read only the pixels that produces a signal and also optional readout capability of the adjacent pixels or channels.
12. Wide range of operation temperatures from −200° C. to 100° C. temperatures. To be designed mainly for room temperature operation.
13. Making the readout ASIC resistant to radiation to increase its lifetime by designing its circuitry using radiation hard design techniques.
14. Fast timing circuitry to add coincident event detection capability.
15. Circuitry for recording both the cathode and the anode signals and using these signals to determine the depth of interaction inside the pixel detector. One technique is to use the ratio of the cathode and anode signals for determining the depth of interaction of the incident particle such as an x-ray or a gamma ray photon.

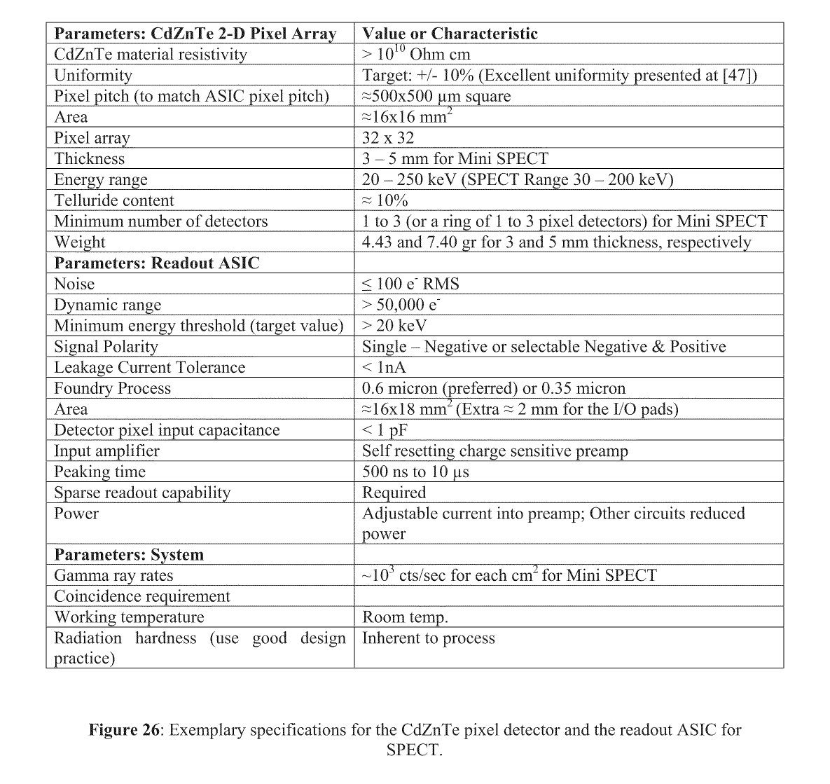

These changes in the chip design demonstrate that the new DANA chip has significant differences compared to the DANA chip. Exemplary initial specifications are set forth in FIG. 26.

Fabrication of the New CZT Pixel Array

New Pixel Geometry

The CZT detector array preferably uses the pixel geometry of FIG. 12. In this geometry, the anode metallization pads are made much smaller in size than the pixel pitch to enhance the small-pixel effect, which suppresses the hole contribution to the induced anode signal. The guard ring on the perimeter of the pixel array is extended around each pixel to form a grid. This grid is used to steer electrons to the central anodes; without it, the enlarged spaces between the smaller anodes would lead to low-field regions in the detector material, resulting in increased charge trapping. The anodes are connected to the charge sensitive input amplifiers of the readout ASIC pixels. The steering grid could be biased with positive or especially negative voltage with respect to the pixel pads to steer the electrons or holes towards the pixel pad(s). This technique and geometry could improve energy resolution significantly.

Charge-Sensitive Input Amplifier

The input amplifier is in many respects the most important section of the readout ASIC. The charge-sensitive amplifier requires large gain so that the small amount of charge generated by the photon interaction inside the detector (˜220 electron-hole pairs per keV) can be amplified and detected with good energy resolution. The small detector signal also implies that the amplifier must have very low noise to achieve a good signal-to-noise ratio (SNR). The best noise performance for charge-sensitive amplifiers can be achieved with pure integrator circuits with only capacitive feedback. The drawback of such circuits is that they are susceptible to baseline drift and saturation due to leakage currents from the detector and need to be reset periodically to restore the original baseline. The charge sensitive amplifier preferably has a resistive feedback in parallel to the capacitive feedback. The feedback capacitance is in the range of a few fF to tens of fF depending on the required dynamic range; for our preliminary design and simulations, we have used a value of 15 fF. The presence of the resistive feedback would cancel the DC signal component which is due to the leakage current, except for a small, constant baseline shift in proportion to the current, and would prevent the baseline from saturating as long as the leakage current stays within reasonable limits. To minimize noise, the feedback resistance needs to be as large as possible. A large resistance limits the leakage current the circuit can tolerate and the bandwidth of the amplifier. In our case, however, the photon rates are not expected to exceed 1,000 per second per pixel, so the bandwidth does not need to be very high in order to avoid pulse pile-up. However, we plan to design for ≈20,000 per second to have the extra room, which is also not difficult to achieve. We plan to use a feedback resistance of 2 GOhm as the best compromise between noise performance and bandwidth. To reduce pile-up, the input amplifier is followed by a differentiator/pole-zero cancellation circuit. We are considering two different ways to implement the resistive feedback circuit.

The first way is to use a resistive multiplier feedback circuit, which is an innovative way to create a large, well-controlled resistor in a relatively small area by using active MOSFET components. This circuit uses current mirror divider circuits configured as an extremely small transconductance amplifier to make an equivalent resistor of several hundred MOhm and up to 2 GOhm, as in our preliminary design. A small resistor on the order of a few hundred kOhm is used as the reference device for this circuit. A measurement of the current going through this reference resistor is made and a new proportional current level of much lower magnitude is driven at the output pin of the transconductance amplifier. The reference resistance is thus effectively multiplied by the inverse of the transconductance amplifier's current gain. This innovative resistive multiplier circuit is essential to achieving such high resistances in a small area because it replaces the large area required for large linear resistors (>10⁸ Ohms) with a compact transistor circuit and much smaller resistor. A resistor of the required size made directly from typical (˜1 kOhm/square) polysilicon would be orders of magnitude larger than the space available for the entire readout channel.

The second way is to use a MOSFET transistor as a resistive feedback element whose resistance is externally controlled. Such circuitry can be used to produce a large resistance feedback component, >10⁹ Ohms, on a much smaller area than even a resistive multiplier circuit. Therefore, it would be especially appropriate to use for smaller pixel sizes such as 250×250 μm² pixel pitch. It can be added as an option to the resistive multiplier circuit so that the two options can be tested and evaluated in comparison to each other. Unfortunately, the MOSFET resistive element is nonlinear and subject to noticeable channel-to-channel variations, and it cannot be used with the pole-zero cancellation circuit.

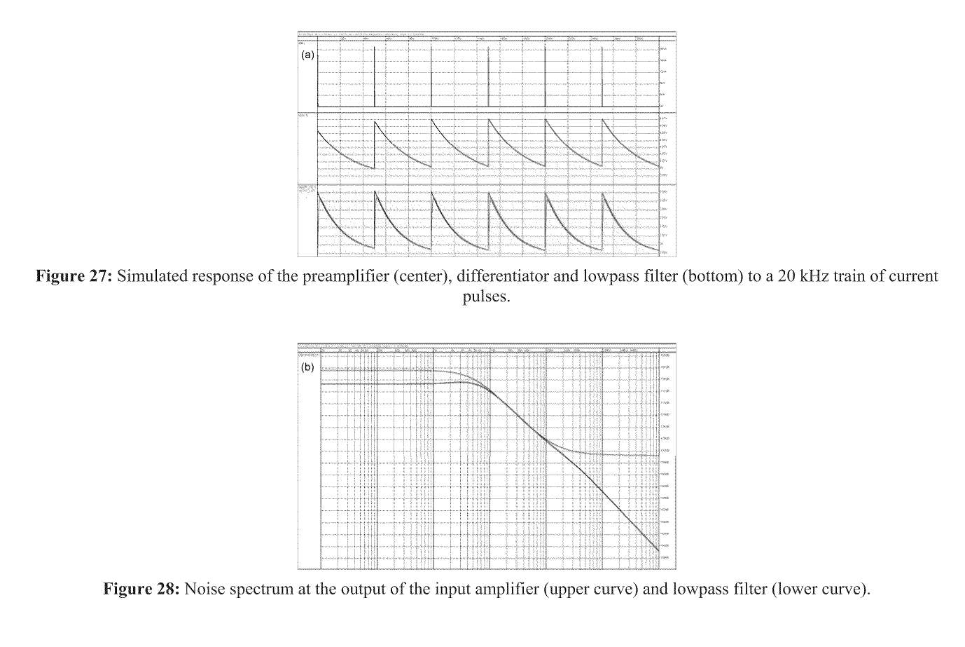

We simulated the behavior of the low-noise, low-bandwidth input amplifier with resistive multiplier feedback of our preliminary design. For the simulation, the amplifier was followed by a differentiator/pole-zero cancellation circuit, which in turn was followed by a lowpass filter/shaper. FIG. 27 shows the responses of these three circuit elements to a series of current pulses generated at a rate of 20 kHz. This rate, which is much higher than the expected photon rate in any pixel, accounts for the random (Poisson-distributed) nature of the photon arrival times. The output signals of the differentiator and shaper do not show any signs of pile-up, even though the preamplifier bandwidth of 5.3 kHz is lower than the pulse rate. FIG. 28 shows the noise spectra at the outputs of the preamplifier and the lowpass filter. The integrated noise at the shaper output is 459 μV rms, for an input-referred value of 51 electrons rms. The simulations were done for a detector capacitance of 1 pF; the corresponding simulation at 0 pF yielded 45 e rms input-referred noise. The pixels of our detectors, flip-chip bonded directly to the readout IC, are expected to have capacitances well below 1 pF, so we expect to reach noise values below 50 e rms.

The size of the resistive multiplier circuit used in these simulations was 0.028 mm², which can be cut in half if we opt not to design the readout channel for selectable signal polarity. Since CZT and most of the other detector materials that might possibly be used with the proposed readout chip have vastly better electron than hole transport properties, having selectable polarities would not contribute much to the chip's usefulness. For comparison, we estimate the size of a MOSFET feedback circuit with similar properties to be 2×10⁻⁴ mm².

Pole-Zero Cancellation Circuit

As discussed above, a standard pole-zero cancellation circuit is preferably used to improve the response at instantaneous count rates that approach or exceed the input amplifier bandwidth. This circuit could restore the baseline and prevent pulse pileup when the count rate approaches these limits. However, due to the low event rate this circuit may not be needed.

Shaper Circuit

The shaper circuit preferably has several different selectable peaking times ranging from ˜0.5 μs to ˜10 microseconds. For example, the peaking time of the shaper used in FIG. 27 was about 2.5 μs. The peaking time selection allows the user to find the optimum balance between energy resolution and count rate for the specific application at hand.

Comparators

A standard comparator circuit could be used to produce an event trigger from the shaper output. The purpose of this trigger signal is preferably to notify external circuitry that an event has been received so that an external process that will read out the ASIC can begin. The triggers could also be recorded into a pixel address (or hit) register for use during the sparse readout. The input amplifier/shaper base line level and gain could be digitally adjustable for each pixel so that a uniform response can be achieved through calibration. The comparator threshold is adjusted globally by a programmable DAC.

Peak Detector

This circuit holds the signal in each pixel at its maximum value until the pulse height is measured and the circuit is reset. This circuit is critical for good energy resolution. If it is not well designed it can produce droop in the output and erroneous pulse height information, which can vary from event to event depending on the order in which channels are read out.

Analog-to-Digital Converter

The peak detector output is preferably digitized directly on the chip. Alternatively, a separate analog-to-digital converter (ADC) on the circuit board or on the microprocessor could be used. This will allow for a more compact system design, as the on-chip ADC can be much smaller than a separately packaged ADC outside the readout IC. Integrating the ADC into the chip also has other advantages, including: no need for an analog output buffer, which tends to be more complex than the digital buffer that would be needed instead; shorter, lower-noise connection between the peak detector and the ADC; input range of the ADC can be matched to the peak detector's output range; and possibly lower overall power dissipation. Since the typical hit multiplicity per event would be low, one ADC for the chip could be sufficient; it could be connected to the appropriate peak detectors under the control of the readout logic.

Sparse Readout

A sparse readout capability is important for 2-D pixel detectors due to the large number of pixels involved. The sparse readout system allows the external circuitry to read out only the pixels, which have generated a trigger. In the proposed applications the majority of the events are expected to trigger a single pixel in a gamma camera and SPECT. Therefore, sparse readout could save significant readout time, data transmission bandwidth, and software overhead. We have already developed sparse readout circuits that function well for our present RENA-2 and RENA-3 ASICs. We plan to implement a similar circuit modified and designed for application to pixel detector readout. In this circuit, the ADC value of the first pixel which has produced a trigger would be connected to the output and its address could be also made available to the outside world. Then, in response to a clock signal, the chip could automatically drop to the second pixel that has produced a trigger, if there is one, and present the output.

Near Neighbor Readout

In a small pixel 2-D detector it may be possible to have charge sharing between adjacent pixels. Since we plan to use 3 to 5 mm thickness and 0.5×0.5 mm pitch charge sharing may not be important. This feature is already built into RENA-2 and RENA-3 chips and works well. One, two or three neighboring pixels may produce a trigger for a single event but the other pixels surrounding them may also have some charge. Therefore, to get the maximum energy resolution it is preferable to read out the charge collected by the neighboring pixels. In this mode, the external circuitry could download the hit register that shows which pixels produced a trigger and then overwrite it with a new pattern that includes the nearest neighbors of the triggered pixels. After this, the readout would proceed as in the sparse case above. Alternatively, for faster readout, the process can start with the sparse readout as above, after which the external circuitry could assert the addresses of additional pixels to retrieve their data. This would eliminate the need to download the entire hit register but require more complex circuitry; for example, the address lines would need to be bi-directional. If charge sharing is not significant the near neighbor mode can be turned off, which not only accelerates the readout but also improves energy resolution, as the noise from the neighbor pixels may outweigh their true energy deposits in these cases. If nearest neighbor readout is appropriate, the energy resolution can be further improved by using advanced reconstruction algorithms rather than summing the signals from all neighbor pixels in all cases.

Other Circuits

A test pulse circuit could be used so that each pixel can be tested by applying and external pulse. The input from this test circuit to each pixel could then be turned on and off individually, which is useful during testing and calibration. A polarity selection circuit may be built into each pixel so that the input to the amplifier can be selected to be electrons or holes. As discussed above, however, the benefit of such circuitry is limited in the case of the pixel detectors for which the chip is designed, and may be outweighed by the increased space and power requirements. The concern is not so much the selection circuit itself but the increased complexity of other circuits, such as the input amplifier, if they need to handle both signal polarities. Preferably, two or more additional full featured analog channels are connected to the chip readout system on the periphery of the ASIC outside the pixel area, with inputs connected to wire bond pads of the ASIC. These analog channels could be mainly used to read out the signals from the detector cathode to determine the depth of interaction of the gamma ray using the pulse height ratio between the anode and the cathode. This ratio would be used to improve the energy resolution of the detector. Another channel will be used to process the signal from the non-collecting grid.

Digital Circuitry

The digital circuits required for the performance of the ASIC such as the setting up, selecting mode of operation, DACs, ADC, and other features could be all external to the pixel area.

Collimator

A parallel fine hole collimator is preferably used when using the proposed detector system as a simple low-energy gamma camera of for SPECT. The thickness of the collimator only needs to be a 2-4 mm to achieve high spatial resolution and low septal penetration because the pixel detector with the collimator could be at touching distance to the small animal's body. In one embodiment, a fine resolution collimator could be used with hole diameter of 0.1 to 0.25 mm diameter. However, smaller or larger hole diameters and collimator thickness may be used without departing from the scope of the current invention. Due to the availability of different types of collimators that can be applied for specific cases the collimator is preferably designed to be easily removable and interchangeable.

Circuit Board Design

The principal tasks that the circuit board for the proposed detector system needs to accomplish are to provide and regulate the power for all electronic circuits, including the detector high voltage; configure, control, and read out the ASIC; and transmit the data to the host computer. The main challenge lies in the design of the circuit board for the self-contained, portable detector system; by comparison, the stationary system imposes less stringent or more easily achievable requirements in terms of power supply, data transmission mode (wireless vs. wired), and allowable board dimensions.

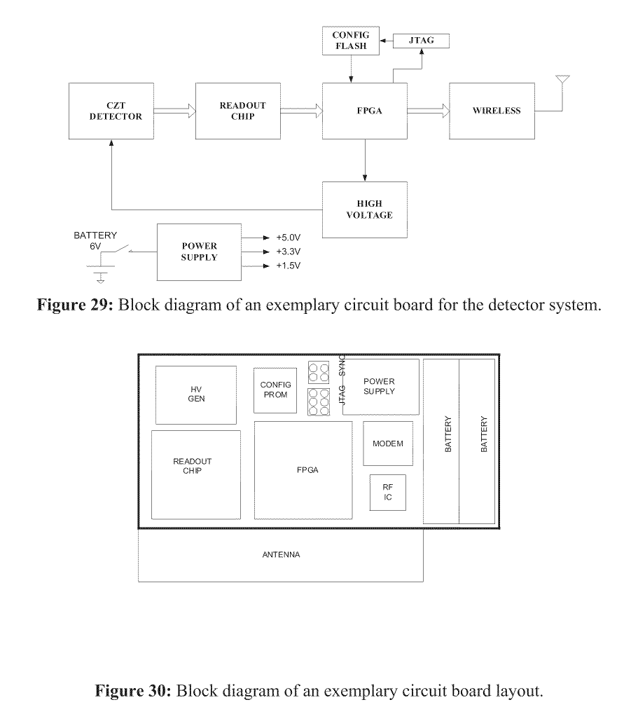

FIG. 29 shows a block diagram of the circuit board for the detector system. The main components of the detector unit are the CZT detector, readout chip, FPGA, wireless module, battery and power supply (regulator). The FPGA combines the data from the readout chip into event messages and sends them to the host PC via the wireless module. The FPGA also controls and configures the readout chip, wireless module, and high voltage module. In another embodiment, the wireless module could be partially implemented with DSP inside the FPGA.

Power Supply

The power for the portable detector system could be supplied by two rechargeable 3 V batteries, for example Toshiba CR2450, a MnO₂/Li coin-type battery with dimensions 24.5 mm dia.×5 mm. The energy output is specified as 620 mAh. This would allow the system to operate for about one hour, sufficient for typical small-animal imaging applications.

The 5 V supply could be provided by a switching regulator with high efficiency, while two low dropout regulators would provide the 3.3 V and 1.8 V, which are expected to require much lower current.

For the detector HV supply, the smallest commercially available solution that we know of is the EMCO Q series of HV generators, with dimensions of (12.7 mm)³. The maximum current that these supplies can provide is much higher than is needed for CZT detectors. A preliminary design of a lower current flyback supply using discrete components indicates that this circuit can be made smaller, 10.5×8×6 mm³, with other specifications similar to the EMCO parts.

Data Transmission

The wireless link communicates between the detector unit(s) and a custom Wireless PCI card residing in the host PC. The transmission range can be very short, 1 m is expected to be a conservative estimate for the maximum range. In the wireless link protocol there are two types of messages, event and time synchronization.

The radio chip set (baseband and RF) could provide the following features in order to assure low bit error rate (BER) per channel:
Sufficient bandwidth to deliver the required data rate. If spreading is required to maintain low BER, the bandwidth should support spreading as well.
Convolutional Code—a type of a long FEC (Forward Error Correcting Code), which can recover from errors.
Bit scrambling to scatter related bits among many blocks so in the event of a potential block failure, the affected bits could be scattered over many code segments, with only a few bits affected in each segment. This could then increase the chance that the FEC will be able to correct the scattered errors.
Capability to use two communication channels.
The wireless link should work within specification with a local Oscillator with Frequency Accuracy of 1 ppm.

We have investigated a number of different options using different chip sets that provide transmission rates between 200 kbps and 310 Mbps (bps: bits per second). Generally, and not surprisingly, the cost, power consumption, and component sizes increase as the data rate increases. An additional solution, as already mentioned, is to implement part of the wireless transmission protocol in the FPGA, reducing size and power compared to other comparable options. The expected data rate capability in this case is 11 Mbps. The data rate could correspond to approximately 30 bits per event if moderate data compression is used. Other suitable wireless technologies could also be used, for example Bluetooth, Wifi, and infrared.

Board Layout

FIG. 30 shows a block diagram of the preliminary circuit board layout, assuming an intermediate size detector of 15×15 mm². Care has been taken to separate the power supplies and wireless module from the detector to avoid noise problems. The board dimensions are 25×50 mm², with the width given by the size of the batteries. If necessary, the board dimensions can be reduced by stacking two circuit boards.

Requirements for Application to Gamma Camera & SPECT

The proposed CdZnTe pixel detector has excellent potential for application to a Mini Gamma Camera and Mini SPECT system for imaging small animals with good quantum efficiency up to ˜150-200 keV (designed to encompass the important 140-keV line from ^{99m} Tc). In these applications, only a very moderate count rate capability of about 10³ cts/sec for each cm² is needed, due to the limited efficiency of the required collimator. A lower energy threshold of a few keV would be especially well suited for imaging radiopharmaceuticals and radiotracers with low energy gamma ray output such as ¹²⁵I.

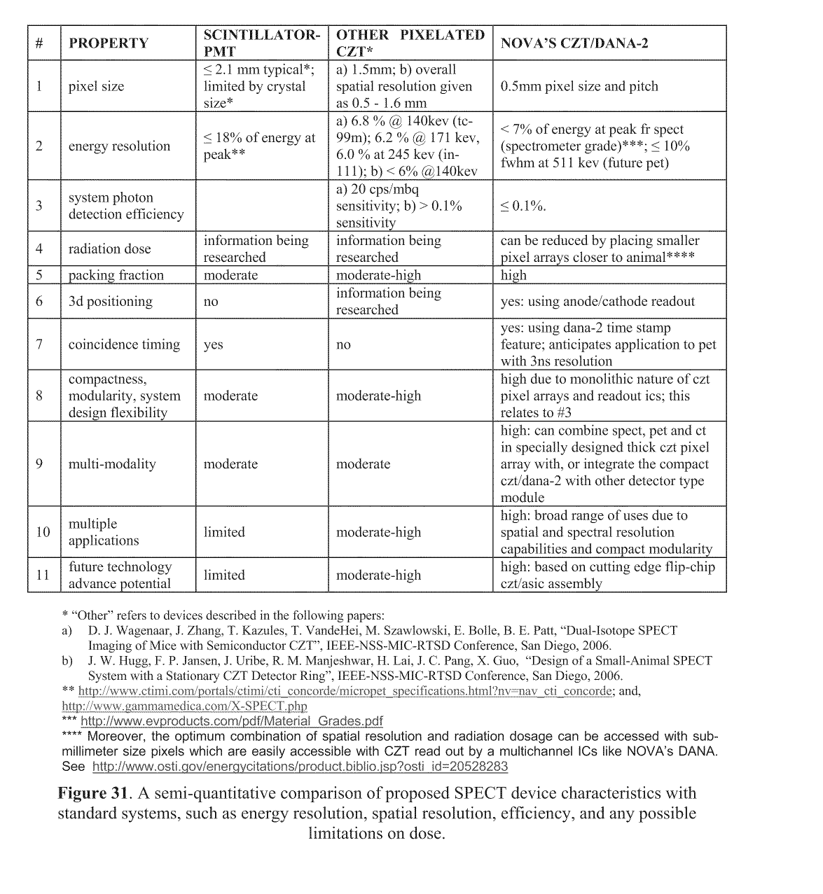

FIG. 31 shows a semi-quantitative comparison of an exemplary SPECT device with those of other devices found in the prior art, such as comparison of energy resolution, spatial resolution, efficiency, and other limitations on dose.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A detection system, comprising:
a harness coupled to a subject, wherein the subject contains a radioactive substance;
a first monolithic pixel detector coupled to the harness that receives a signal from the radioactive substance; and
an integrated circuit coupled to the first monolithic pixel detector to process the signal and produce an image from the signal, wherein a weight of the first monolithic pixel detector and the integrated circuit is supported by the harness.

2. The detection system of claim 1, wherein the subject comprises an animal and wherein the harness comprises a helmet that wraps around a portion of a head of the animal.

3. The detection system of claim 1, wherein the first monolithic pixel detector comprises a collimator.

4. The detection system of claim 1, further comprising a wireless transceiver that transmits the image to a distal wireless receiver.

5. The detection system of claim 1, further comprising a storage memory that receives the image.

6. The detection system of claim 5, wherein the storage memory is detachable from the detection system.

7. The detection system of claim 5, further comprising a port that couples to the storage memory to transmit the image to a device coupled to the port.

8. The detection system of claim 1, wherein the first monolithic pixel detector is flip-chip bonded to the integrated circuit.

9. The detection system of claim 1, wherein the first monolithic pixel detector comprises a plurality of channels that receive the signal from the radioactive substance.

10. The detection system of claim 1, wherein the signal comprises a photon resulting from an annihilation of an electron and a positron.

11. The method of claim 1, further comprising providing power to the detection system wirelessly.

12. The detection system of claim 1, wherein the first monolithic pixel detector comprises two monolithic pixel detectors placed in close proximity of each other.

13. The detection system of claim 1, wherein a second monolithic pixel detector is placed opposing the monolithic pixel detector.

14. The detection system of claim 13, wherein at least one of the first and second monolithic pixel detectors comprises two monolithic pixel detectors placed in close proximity of each other.

15. A method of imaging a subject; comprising:
introducing a radiation-producing substance to the subject;
coupling a first monolithic pixel detector to the subject that receives a signal from the photon-producing substance, wherein the first monolithic pixel detector is coupled to an integrated circuit, wherein the subject supports the weight of the first monolithic pixel detector and the integrated circuit;
processing the signal with the integrated circuit to produce an image; and
outputting the image.

16. The method of claim 15, wherein the subject is an animal and wherein the first monolithic pixel detector is mounted to a harness adapted to couple with the animal.

17. The method of claim 16, wherein the harness comprises a helmet.

18. The method of claim 15, wherein the step of outputting the image comprises wirelessly transmitting the image to an external device, wherein the external device is not physically coupled to the first monolithic pixel detector.

19. The method of claim 15, wherein the step of introducing a radiation-producing substance to the subject comprises injecting a radiopharmaceutical to a portion of the subject.

20. The method of claim 19, wherein the radiopharmaceutical produces a positron that annihilate with an electron to produce a photon that composes a portion of the signal.

21. The method of claim 15, wherein the step of introducing a radiation-producing substance to the subject comprises coupling a radiation-producing device to an opposing side of the subject facing the first monolithic pixel detector.

22. The method of claim 15, further comprising producing a plurality of images from a plurality of signals received by the first monolithic pixel detector.

23. The method of claim 15, further comprising flip-chip bonding the first monolithic pixel detector to the integrated circuit.

24. The method of claim 15, wherein the monolithic pixel detector comprises two monolithic pixel detectors placed in close proximity of each other.

25. The method of claim 15, wherein a second monolithic pixel detector is placed opposing the monolithic pixel detector.

26. The method of claim 25, wherein at least one of the first and second monolithic pixel detectors comprise two monolithic pixel detectors placed in close proximity of each other.

27. The detection system of claim 1, wherein a surface area of a receiving side of the first monolithic pixel detector is at most 32×32 mm².

28. The method of claim 15, wherein a surface area of a receiving side of the first monolithic pixel detector is at most 32×32 mm².
